(19) United States
(12) Patent Application Publication
(10) Pub. No.: US 2002/0034479 A1
(43) Pub. Date: Mar. 21, 2002

(54) Composition

(75) Inventor: Alison Katharine Green, Bebington (GB)

Correspondence Address: UNILEVER, PATENT DEPARTMENT, 45 RIVER ROAD, EDGEWATER, NJ, 07020, US

(73) Assignee: Unilever Home & Personal Care USA, Division of Conopco, Inc.

(21) Appl. No.: 09/923,522

(22) Filed: Aug. 7, 2001

(30) Foreign Application Priority Data
Aug. 7, 2000 (EP) 00306726.1

(51) Int. Cl. A61K 7/16; A61K 7/18

(52) U.S. Cl. 424/52

(57) ABSTRACT

Oral composition comprising petroleum jelly in the form of droplets enrobing a particulate active, characterised in that the droplets comprise an amphiphilic organic material, capable of forming, upon contact with moisture, a water-insoluble liquid crystal phase of at least one dimensional periodicity.

The present invention relates to a composition according to the preamble of claim 1.

Modern dental hygiene products typically contain ingredients included to provide a benefit to the consumer. Such ingredients include antimicrobial agents, e.g. Triclosan; anti-caries agents, e.g. fluoride and flavours such as peppermint extract to name but a few.

An obvious problem with including agents in oral preparations is that within a short period of time much of the agent is removed from the oral cavity through the action of rinsing or because of the increased salivation in response to something being put into the oral cavity. The fact that the average consumer brushes approximately once every twelve hours only exacerbates the problem of sustaining enough agent to have an effect.

Unsurprisingly there has always been a need for an oral composition which is capable of prolonging the retention of such ingredients.

An additional problem is that some ingredients cannot be used successfully in oral care compositions because they react with other commonly used ingredients. For example, sodium fluoride is the most popular anti-caries agent in oral care technology but it cannot be used with a composition which comprises as abrasive chalk. The calcium fluoride which is formed is a poor source of fluoride ions and the result is a poor anti-caries effect.

One of the common ways to include in an oral composition ingredients which interact with one another is by separating them with expensive packaging technology. However, the expense alone requires that improvements are required in formulation technology.

Another way of separating ingredients in the same formulation, i.e. without the need for expensive packaging, is the use of breakable capsules. For example, GB 1 381 444 (Blendax) describes typical capsules which can be used in an oral composition. The downside of capsules is that if they are too soft they may break during manufacture and yet if they are too hard they may be felt by the consumer or even that they do not break at all during brushing. A further disadvantage of capsules is that they are expensive.

Petroleum jelly is an ingredient which has been proposed for use in oral compositions in the patent literature. However, its use has only ever been suggested as a mere excipient and not for anything substantially relating to the efficacy of any of the other ingredients.

An example of the prior art is US 4 098 878 (Colgate) in which it is disclosed that petroleum jelly is a suitable non-polar waxy material in an oral composition.

Despite all that is available in the prior art with respect to the use of agents to improve the efficacy of a particular active or for the use of petroleum jelly in an oral composition we have surprisingly found that by putting petroleum jelly in an oral composition in the form of droplets which themselves enrobe an active agent the efficacy of said active can be prolonged.

One of the merits of petroleum jelly is that it is cheap in comparison to many of the other ingredients used in oral care technology.

A further advantage of petroleum jelly is that it is capable of compartmentalising a hydrous formulation so that actives which could not be formulated together because of cross-reactivity may now be included in the same formulation by enrobing one of them in petroleum jelly.

Yet a further advantage of petroleum jelly, particularly over capsules, is that the droplets of petroleum jelly are much more likely to survive manufacturing processing and still able to deliver the active. Moreover, unlike a capsule which has to deliver the active during brushing the petroleum jelly droplets may stick to the surfaces of the oral cavity and release the active over time.

Accordingly, the invention provides an oral composition according to claim 1.

Petroleum jelly is commercially available from numerous sources. Examples of commercially available petroleum jellies include: Merkur and Vara series from Merkur Vaseline, Astor M0140 from Allied Signal, White Fonoline H from Witco, Silkolene series from Fuchs Lubricants, Penreco series ex Penreco.

It is an essential feature of the invention that the petroleum jelly is in the form of droplets and that at least part of these droplets enrobe an active agent. The function of the petroleum jelly is to enhance the effect of this active. This can be achieved by maintaining it in the oral cavity until long after brushing has ceased or even as a result of its localisation away from other ingredients in the formulation.

Amphiphilic organic material as herein defined is an organic material which has both hydrophobic and hydrophilic portions in its structure.

When water and certain water-insoluble or sparingly water-soluble organic materials are brought together, the organic materials pass through various physical phases upon the addition of water. They can form, in their final state in the aqueous medium liquid crystal structures of various dimensional periodicities. This is more fully described e.g. in “Biological Membranes” by D. Chapman, Academic Press New York, 1968, Chapter 3, and in “Nature”, Vol. 222, page 1159 (1969) by Balmbra et al.

The amphiphilic organic materials according to the present invention must be capable of forming, upon addition of water or moisture (e.g. saliva), in their final state a liquid crystal phase of at least one-dimensional periodicity, e.g. a lamellar phase. Preferably, they should be capable of so forming a liquid crystal phase with a two-dimensional periodicity, e.g. a hexagonal phase, and particularly preferably they should be capable of so forming a liquid crystal phase with a three-dimensional periodicity, e.g. a cubic phase.

The use of such amphiphilic materials for certain applications has already been described in the art. Thus, in WO-A-84/02076 (Fluid-Carbon International AB), controlled-release compositions for biologically-active materials are described, comprising amphiphilic organic materials, capable of forming liquid crystalline phases, in which the biologically-active material is either dissolved or dispersed, or is coated by the liquid crystalline phase. These compositions are stated to be suitable for controlled release of medication and drug delivery.

In “Pharmaceutical Technology Europe” February 1995, pages 14-17, Engström et al. describe similar systems for drug delivery through the oral mucosa, exemplified by the glyceryl monooleate-water system.

The amphiphilic organic material, suitable for use in the present invention can be selected from a host of materials, known in the art, e.g. as described in the above-mentioned references. They should, of course, be compatible with the other components of a toothpaste, and should meet the requirements of safety, taste, colour etc. that are usually set for ingredients of a toothpaste. Typical suitable examples are unsaturated and/or saturated C₁₂-C₂₄ fatty acid glycerides, optionally in admixture with long chain fatty acids and/or fatty alcohol and/or polyalkylene glycols such as glyceryl monooleate, optionally in admixture with oleic acid, glyceryl monolaurate, in admixture with oleic acid or with oleyl alcohol, stearyl alcohol, isostearyl alcohol or a mixture thereof; glyceryl mono-isostearate, glyceryl mono-linoleate in admixture with glyceryl mono-oleate, polyoxyethylene ethers, mixtures of lecithin and oleic acid or oleyl alcohol, mixtures of sodium or potassium oleate with oleic acid or oleyl alcohol, certain silicone materials such as sodium 10-Ω-butyl [poly(dimethylsiloxy) dimethyl silyl] decanoate. Mixtures of any of these materials may also be used.

The preferred amphiphilic materials are the lipid substances, i.e. the fatty acid glycerides, in particular glyceryl monooleate, glycerylmonolinoleate, glyceryl monoisostearate, optionally in admixture with a fatty acid or fatty alcohol, and the particularly preferred material is glyceryl monooleate (GMO). In this respect it is observed, that in EP-A-429,224 a composition, suitable for insertion into or around the periodontal pocket of a person suffering from diseases of the oral cavity is described, which composition comprises monoolein (=GMO) and a drug active. The drug active can be selected from a great variety of different drug actives, among which dentinal desensitising agents such as strontium chloride or sodium fluoride are mentioned. The GMO according to this reference absorbs water from the body fluid surrounding the periodontal cavity and becomes more viscous, enabling extended duration of retention at the site of treatment, releasing the drug active in a controlled manner slowly over extended duration.

The amphiphilic agent is generally used in the droplet in an amount of between 1 and 60% by weight, preferably between 5 and 45% by weight and particularly preferably between 10 and 40% by weight of the toothpaste.

The particulate active may be any active which can provide a benefit in the oral cavity and which is capable of being present in particulate form whether by spray-drying or otherwise. Examples of actives include:

antimicrobial agents, e.g. Triclosan, chlorhexidine, copper-, zinc- and stannous salts such as zinc citrate, zinc sulphate, zinc glycinate, sodium zinc citrate and stannous pyrophosphate, sanguinarine extract, metronidazole, quaternary ammonium compounds, such as cetylpyridinium chloride; bis-guanides, such as chlorhexidine digluconate, hexetidine, octenidine, alexidine; and halogenated bisphenolic compounds, such as 2,2′ methylenebis-(4-chloro-6-bromophenol);

anti-inflammatory agents such as ibuprofen, flurbiprofen, aspirin, indomethacin etc.;

anti-caries agents such as sodium- and stannous fluoride, aminefluorides, sodium monofluorophosphate, sodium trimeta phosphate and casein;

plaque buffers such as urea, calcium lactate, calcium glycerophosphate and strontium polyacrylates;

vitamins such as Vitamins A, C and E;

plant extracts;

anti-sensitive teeth agents, e.g. potassium citrate, potassium chloride, potassium tartrate, potassium bicarbonate, potassium oxalate, potassium nitrate and strontium salts;

anti-calculus agents, e.g. alkali-metal pyrophosphates, hypophosphite-containing polymers, organic phosphonates and phosphocitrates etc.;

H2-antagonists, e.g. cimetidine and ranitidine;

anti-oxidants;

biomolecules, e.g. bacteriocins, antibodies, enzymes, etc.; and

flavours, e.g. peppermint and spearmint oils;

While it is quite possible for the active to be any which has been postulated for use in oral care before, even those which have never made it to the marketplace, it is preferable that the active is at least partially water soluble or partially water absorbent. More preferably the active is substantially water soluble or substantially water absorbent. Even more preferably the active is one which may exist in a particulate, e.g. crystalline or powder form.

A particularly useful active according to the present invention is a fluoride ion source such as sodium fluoride.

It is also within the ambit of the present invention that the active comprises an independent active delivery system. For example, the active could comprise an active agent which is merely being separated from the remainder of the composition by the petroleum jelly. An example of such would be polyacrylic acid and triclosan in the form of spray-dried spheres. The particles are enrobed by the petroleum jelly and are separated from the remainder of the composition, which more usually comprises water. During brushing the particles are dispersed around the oral mucosa and are ruptured to release the particulate matter. These polyacrylic acid particles become unstable in the presence of water thereby releasing the triclosan. This system has the further advantage that an active which is at least partially soluble in the petroleum jelly can be used in the present invention without just being dissolved in the petroleum jelly.

Similarly useful materials which may be used as binders include carboxymethyl cellulose, gelatin and gum arabic to name a few.

A further example comprises petroleum jelly droplets comprising an active which is unstable at higher pH ranges. Formulations comprising chalk abrasives often have a pH as high as 10.5. If the active is unstable at pH 10.5 it could be enrobed in petroleum jelly where it will be released only during brushing or after brushing has ended and, more importanly, when the pH of the oral cavity is neutral.

The size of the droplets is not thought to be of importance but it is necessary for them to be sufficiently large to fully enrobe the active. It is also thought that droplets any bigger than 2 mm in diameter would be clearly visible in the oral cavity after brushing and this may be a significant consumer negative. It may be that any individual droplet can enrobe a plurality of active particles or even a plurality of different actives. The droplets can also be coloured using technology known in the art.

In a preferred embodiment the composition according to the invention comprises a wetting agent. Suitable wetting agents include surfactants such as the anionic, cationic, non-ionic and zwitterionic surfactants. Preferred wetting agents include the anionic surfactants such as the alkali-metal alkyl sulphates, e.g. sodium lauryl sulphate (SLS). Where present the wetting agent is in the composition in an amount ranging from 0.01 to 5% by weight of the composition, preferably from 0.1 to 3.5% by weight and especially from 0.75 to 2.5% by weight of the composition.

The oral composition according to the invention comprise further ingredients which are common in the art, such as those already disclosed as possible actives above. Examples include:

proteinaceous materials such as collagen and keratin;

preservatives;

opacifying agents;

colouring agents;

pH-adjusting agents;

sweetening agents;

pharmaceutically acceptable carriers, e.g. starch, sucrose, water or water/alcohol systems etc.;

surfactants, such as anionic, nonionic, cationic and zwitterionic or amphoteric surfactants;

particulate abrasive materials such as silicas, aluminas, calcium carbonates, dicalciumphosphates, calcium pyrophosphates, hydroxyapatites, trimetaphosphates, insoluble hexametaphosphates and so on, including agglomerated particulate abrasive materials, usually in amounts between 3 and 60% by weight of the oral care composition.

humectants such as glycerol, sorbitol, propyleneglycol, xylitol, lactitol etc.;

binders and thickeners such as sodium carboxymethyl-cellulose, xanthan gum, F127, gum arabic etc. as well as synthetic polymers such as polyacrylates and carboxyvinyl polymers such as Carbopol®;

polymeric compounds which can enhance the delivery of active ingredients such as antimicrobial agents can also be included. Examples of such polymers are copolymers of polyvinylmethylether with maleic anhydride and other similar delivery enhancing polymers, e.g. those described in DE-A-3,942,643 (Colgate);

buffers and salts to buffer the pH and ionic strength of the oral care composition; and

other optional ingredients that may be included are e.g. bleaching agents such as peroxy compounds e.g. potassium peroxydiphosphate, effervescing systems such as sodium bicarbonate/citric acid systems, colour change systems, and so on.

Liposomes may also be used to improve delivery or stability of active ingredients.

Clearly any of these further materials may also be used along with the active within the petroleum jelly droplet.

The oral compositions may be in any form common in the art, e.g. toothpaste, gel, mousse, aerosol, gum, lozenge, powder, cream, etc. and may also be formulated into systems for use in dual-compartment type dispensers.

Since the droplets according to the invention comprise an active it is also possible to tailor a release profile to attain a particular end benefit. For example, if sodium fluoride were to be incorporated into droplets as hereinbefore described most of the fluoride would be released only after brushing. It may also be important to have some fluoride present at the beginning and during brushing. Thus, it may be preferred to include some fluoride in the base composition as well as in the droplets. In this way there is an immediate release of fluoride in combination with a delayed release.

Such tailoring could also be used to provide different sensory aspects to brushing. For example, it may be possible to provide, within the base of the composition, a particular flavour ingredient while having a complementary flavour in the droplets. The former provides an immediate sensory effect while the latter may provide a complementary longer lasting effect.

Using this kind of strategy it would be obvious to one skilled in the art of oral composition manufacture to provide any of a number of different actives in various combinations according to the invention to achieve an improved benefit.

A composition according to the invention is made by pre-mixing the petroleum jelly with the particulate active and the amphiphilic organic material and then incorporating this mixture into an oral composition base. The base usually comprises the remaining materials to be included in the composition although it is also possible for further materials to be included after the petroleum jelly/active mix has been added. The composition is then mixed to form the droplets of petroleum jelly and active. Preferably, the petroleum jelly is mixed with the crystalline form of the active.

Embodiments of the invention will now be described with reference to the following non-limiting examples:

### EXAMPLE 1

The following is a formulation according to the invention:

| Ingredient | % w/w |
|---|---|
| Abrasive silica | 10.00 |
| Sorbitol (70%) | 45.00 |
| Sodium lauryl sulphate | 1.50 |
| Sodium carboxymethyl cellulose | 0.90 |
| Thickening silica | 8.00 |
| Sodium fluoride | 0.32 |
| Water | 24.11 |
| Flavour | 1.00 |
| Sodium saccharin | 0.17 |
| Polyethylene glycol | 5.00 |
| Titanium dioxide | 1.00 |
| Petroleum jelly droplet containing sodium | 3.00 |
| fluoride and glyceryl mono isostearate | |

### EXAMPLE 2

The following is a method for the manufacture of a formulation according to example 1.

Manufacture base composition using standard methods. Disperse active (sodium fluoride crystals) into petroleum jelly. Add petroleum jelly/sodium fluoride mix to amphiphilic material and then to base composition and mix thoroughly.

1. Oral composition comprising petroleum jelly in the form of droplets enrobing a particulate active, characterised in that the droplets comprise an amphiphilic organic material, capable of forming, upon contact with moisture, a water-insoluble liquid crystal phase of at least one dimensional periodicity.

2. Oral composition according to claim 1, characterised in that the active is at least partially soluble in water.

3. Oral composition according to claim 1, characterised in that the active is selected from the group consisting of anti-caries agents, anti-plaque agents, anti-microbial agents, anti-sensitive teeth agents, flavours, H-2 antagonists, teeth whitening agents, anti-inflammatory agents, phosphates, nutrients, enzymes, anti-oxidants and mixtures thereof.

4. Oral composition according to claim 1, characterised in that the active is a fluoride ion source.

5. Oral composition according to claim 1, characterised in that the active is sodium fluoride.

6. Oral composition according to claim 1, characterised in that it comprises from 0.01 to 50% by weight petroleum jelly.

7. Oral composition according to claim 1, characterised in that it comprises from 0.1 to 10% by weight petroleum jelly.

8. Oral composition according to claim 1, characterised in that the composition comprises a wetting agent.

9. Oral composition according to claim 8, characterised in that the wetting agent is an alkali-metal alkyl sulphate.

10. Oral composition according to claim 1, wherein the amphiphilic organic material is a C₁₂-C₂₄ unsaturated and/or saturated fatty acid glycerides.

11. Oral composition according to claim 1, wherein the amphiphilic organic material is glyceryl monooleate, glyceryl monolinoleate, glyceryl monoisostearate or mixtures thereof.

12. Oral composition according to claim 1, wherein the amphiphilic organic material is a material, capable of forming, upon contact with moisture, a hexagonal phase.

13. Oral composition according to claim 1, wherein the amphiphilic organic material is a material, capable of forming, upon contact with moisture, a cubic phase.
